(19) United States
(12) Patent Application Publication
(10) Pub. No.: US 2007/0196914 A1
(43) Pub. Date: Aug. 23, 2007

(54) Methods for preparing oil bodies comprising active ingredients

(75) Inventors: Elizabeth Wanda Murray, Calgary (CA); Joseph Boothe, Calgary (CA); Nancy-Ann Markley, Calgary (CA)

Correspondence Address: BERESKIN AND PARR, 40 KING STREET WEST, BOX 401, TORONTO, ON, M5H 3Y2, CA

(73) Assignee: SemBioSys Genetics Inc., Calgary, AB (CA)

(21) Appl. No.: 10/572,591

(22) PCT Filed: Oct. 4, 2004
(86) PCT No.: PCT/CA04/01792

§ 371 Date: Dec. 14, 2006

Related U.S. Application Data
(60) Provisional application No. 60/507,505, filed on Oct. 2, 2003.

(51) Int. Cl. A61K 31/685; A61K 9/127; A61K 31/24

(52) U.S. Cl. 435/325; 435/243; 435/410; 424/450; 514/28; 514/536; 514/78

(57) ABSTRACT

The present invention provides novel emulsions that comprise oil bodies. The invention also relates to novel methods for generating formulations comprising oil bodies and active ingredients wherein the active ingredient is partitioned into the oil body. The methods are particularly useful for generating emulsions with either hydrophobic or amphipathic biologically active agents.

### FIELD OF THE INVENTION

The present invention provides novel emulsions that comprise oil bodies. The invention also relates to novel methods for generating formulations comprising oil bodies and active agents wherein the active ingredient is partitioned into the oil body. The methods are particularly useful for generating emulsions with either hydrophobic or amphipathic biologically active agents.

### BACKGROUND OF THE INVENTION

In the seeds of oilseed crops, which include economically important crops, such as soybean, rapeseed, sunflower, safflower and palm, the water insoluble oil fraction is stored in discrete subcellular structures variously known in the art as oil bodies, oleosomes, lipid bodies or spherosomes (Huang 1992, Ann. Rev. Plant Mol. Biol. 43: 177-200). Besides a mixture of oils (triacylglycerides), which chemically are defined as glycerol esters of fatty acids, oil bodies comprise phospholipids and a number of associated proteins, collectively termed oil body proteins. From a structural point of view, oil bodies are considered to be a triacylglyceride matrix encapsulated by a monolayer of phospholipids in which oil body proteins are embedded (Huang, 1992, Ann. Rev. Plant Mol. Biol. 43: 177-200). The seed oil present in the oil body fraction of plant species is a mixture of various triacylglycerides, of which the exact composition depends on the plant species from which the oil is derived. It has become possible through a combination of classical breeding and genetic engineering techniques, to manipulate the oil profile of seeds and expand on the naturally available repertoire of plant oil compositions. For an overview of the ongoing efforts in his area, see Designer Oil Crops/Breeding, Processing and Biotechnology, D. J. Murphy Ed., 1994, VCH Verlagsgesellschaft, Weinheim, Germany.

Plant seed oils are used in a variety of industrial applications. In order to obtain the plant oils used in these applications, seeds are crushed or pressed and subsequently refined using processes such as organic extraction, degumming, neutralization, bleaching and filtering. Aqueous extraction of plant oil seeds has also been documented (for example, Embong and Jelen, 1977, Can. Inst. Food Sci. Technol. J. 10: 239-243). Since the objective of the processes taught by the prior art is to obtain pure oil, oil bodies in the course of these production processes lose their structural integrity. Thus, the prior art emulsions formulated from plant oils generally do not comprise intact oil bodies.

U.S. Pat. No. 5,683,740 to Voultoury et al. and U.S. Pat. No. 5,613,583 to Voultoury et al. disclose emulsions comprising lipid vesicles that have been prepared from crushed oleagenous plant seeds. In the course of the crushing process described in these patents, oil bodies substantially lose their structural integrity. Accordingly, it disclosed that in the crushing process, 70% to 90% of the seed oil is released in the form of free oil. Thus the emulsions, which are the subject matter of these patents, are prepared from crushed seeds from which a substantial amount of free oil has been released while the structural integrity of the oil bodies is substantially lost. In addition, the emulsions disclosed in both of these patents are prepared from relatively crude seed extracts and comprise numerous endogenous seed components including glycosylated and non-glycosylated non-oil body seed proteins. It is a disadvantage of the emulsions to which these patents relate that they comprise contaminating seed components imparting a variety of undesirable properties, which may include allergenicity and undesirable odour, flavour, colour and organoleptic characteristics, to the emulsions. Due to the presence of seed contaminants, the emulsions disclosed in these patents have limited applications.

A non-destructive preparation of oil bodies is disclosed by Deckers et al. (U.S. Pat. Nos. 6,183,762, 6,210,742, 6,146,645, 6,372,234, 6,582,710, 6,582,710, 6,596,287, 6,761,914, US 2002100373036, and U.S. Pat. No. 6,599,513). In accordance with these patents and patent applications a purified preparation of oil bodies is collected as a natural emulsion and further emulsions may be prepared in the presence of a multiplicity of other substances in order to achieve a desirable balance of emulsification, viscosity, stability and appearance in order to render the emulsions suitable for inter alia cosmetic, pharmaceutical and food applications. Additional ingredients to achieve these characteristics may include water, emulsifiers, stabilizers, thickening or thinning agents, preservatives, fragrances or other additives. Of particular interest are oil body preparations containing active ingredients. While simply mixing the active ingredient of interest with the oil body emulsion may result in an acceptable oil body preparation, it is particularly desirable to prepare oil body emulsions comprising active ingredients which partition selectively with the oil body. For example, traditionally unstable actives may be stabilized when partitioned into the core of an oil body. Furthermore, in oil body formulations which are used for topical application to the human skin, the delivery characteristics of the active ingredient to the human skin may be modulated when the active is partitioned into the oil body. While simple mixing in accordance with the above mentioned Deckers patents may promote some partitioning of the active ingredient, frequently no partitioning is achieved or the partitioning of the active ingredient is sub-optimal. To be considered partitioned, actives must be in physical contact with the oil body for example by bonding or some other affiliation and must partition with the oil body.

Thus, there is a need in the art for facilitating the partitioning of actives, including for example, traditionally unstable actives, into intact oil bodies.

### SUMMARY OF THE INVENTION

The herein mentioned invention provides for one or more hydrophobic and/or amphipathic actives of interest to be partitioned into the internal oil core, onto the lipid membrane, into the lipid membrane or attached to the external surface of the lipid membrane of the oil body.

Herein described is a new system for improved oil body partitioning. The system involves the use of two-solvents and solubilization of an active resulting in the blending of the active and the oil body emulsion. The system is more complex than mixing the oil body emulsions with the active ingredients and results in increased active partitioning onto or into the oil bodies. This is especially useful for partitioning solid and semi-solid, hydrophobic and amphipathic molecules that are particularly difficult to solubilize, often requiring the use of organic solvents. Removal of the first solvent can be performed in an optional step when this solvent is incompatible or undesirable in the final product. Finally, the active and solvents are blended and partitioned into the oil bodies.

The present invention relates to novel methods for generating formulations comprising oil bodies and active agents wherein the active agent is partitioned into the oil body. Presently, the inventors have discovered novel methods for preparing oil bodies comprising active agents, including actives that are reactive and unstable in current formulations. Broadly stated, the present invention provides methods for formulating emulsions containing active agents partitioned into oil bodies, wherein they are stabilized and readily available for topical or oral delivery.

Accordingly, the present invention provides a method of partitioning an active agent into oil bodies comprising:
a) dissolving the active agent in a first solvent;
b) mixing the dissolved agent with a second solvent; and
c) contacting the solvent mixture with oil bodies to partition the active agent into the oil bodies.

In a preferred embodiment of the invention, the active agent does not partition into oil bodies when contacted with the oil bodies in the absence of a solvent or when the active agent is dissolved in the first solvent. In a further preferred embodiment of the invention, the active agent is selected from the group of active agents consisting of hydrophobic molecules and amphipathic molecules.

In a further preferred embodiment of the present invention, the first solvent is an organic solvent. Preferably, the first solvent is substantially removed by evaporation or substantially reduced in volume by dilution after mixing with the second solvent.

In yet a further preferred embodiment of the invention, the second solvent is selected from the group of solvents consisting of water, aqueous buffer, oils, fatty acids, and lipids.

The methods of preparing oil bodies comprising active agents, and the resulting emulsions of the present invention can be used in a wide range of applications including in the preparation of personal care and dermatological products. Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### DETAILED DESCRIPTION OF THE INVENTION

As hereinbefore mentioned, the present invention relates to novel methods for generating formulations comprising oil bodies and active agents. The invention also relates to the novel emulsion formulations that are prepared from oil bodies. Using the present invention, it is possible to partition an active ingredient, for example 20-30% (dry weight active/dry weight of oil body), into oil bodies. The inventors have also found that certain biologically active agents that are reactive and unstable in current formulations, are made stable and thereby more effective as personal care and dermatological products. The level of loaded active achievable, the stabilizing properties of the oil bodies, and the ability to topically deliver novel agents make the present invention of considerable use.

Accordingly, pursuant to the present invention a method for the partitioning of active agents into oil bodies is provided in which the method comprises:
(i) dissolving the active agent in a first solvent;
(ii) mixing the dissolved active agent with a second solvent to obtain a mixture of the first and second solvent comprising the active agent; and
(iii) contacting said mixture of the first and second solvent with oil bodies to partition said active agent into said oil bodies.

In a preferred embodiment said active agent does not partition into oil bodies when contacted with oil bodies in the absence of a solvent or when the active agent is directly dissolved in the first solvent. Preferably the active agent is further characterized in that the active agent is insoluble, or essentially insoluble in water. The methods of the present invention are particularly useful when the active agent additionally is insoluble or essentially insoluble in the second solvent

The terms “partition”, “partitioning” and “partitioned” as used herein mean the active is located in the internal oil core, onto the lipid membrane, into the lipid membrane or attached to the external surface of the lipid membrane of the oil body.

Solvents

The term “first solvent” as used herein refers to the first or initial solvent that is used to dissolve the active agent. Preferably the first solvent is an organic solvent. Examples of organic solvents, include but are not limited to, alcohols, aliphatic hydrocarbons, aromatic hydrocarbons, chlorinated hydrocarbons, glycols, glycol ethers and their acetates, esters, ethers and ketones. Examples of alcohols include, but are not limited to, methanol, ethanol and isopropyl alcohol (isopropanol). Examples of aliphatic hydrocarbons include, but are not limited to n-hexane. Examples of aromatic hydrocarbons include, but are not limited to toluene, xylene, styrene and benzene. Examples of chlorinated hydrocarbons include, but are not limited to perchloroethylene, methylene chloride, carbon tetrachloride, methyl chloroform, chloroform, and trichloroethylene. Examples of glycols include, but are not limited to, propylene glycol, triethylene glycol, and ethylene glycol. Examples of glycol ethers include, but are not limited to butyl cellusolve (2-butoxyethanol), cellusolve (2-ethoxyethanol), methyl cellusolve (2-methoxyethanol), and cellusolve acetate (2-ethoxyethyl acetate). Examples of esters include, but are not limited to methyl formate, ethyl acetate, isopropyl acetate, methyl acetate, secamylacetate, and isoamyl acetate. Examples of ethers include, but are not limited to ethyl ether, tetrahydrofuran, dioxane and isopropyl ether. Examples of ketones include, but are not limited to, acetone, methyl ethyl ketone (MEK), cyclohexanone and isophorone. More preferably the first solvent is an alcohol or a chlorinated hydrocarbon. Most preferably the first solvent is selected from the group of solvents consisting of isopropanol, ethanol and chloroform. In addition, the first solvent could be an oil, lipid or fatty acid.

The term “second solvent” as used herein refers to the solvent that is mixed with the active agent once it is dissolved in the first solvent. The second solvent can be any solvent that is compatible with oil bodies. Note that the methods of the present invention are particularly useful in instances where the active agent is not directly soluble in oil bodies or the second solvent. The solubility of the active agent in the second solvent can be readily determined by a person ordinarily skilled in the art, for example by referring to Standard Chemical Indices which provide information on stability, including, but not limited to, The CRC, Handbook of Chemistry and Physics or The Merck Index, Merck & Co., Inc. Budavari S (Ed.). The second solvent is selected from a group consisting of water, aqueous buffer, oils, fatty acids, and lipids. Examples of aqueous buffers include but are not limited to buffers containing phosphate, phosphate buffered saline, bicarbonate and HEPES (N-[2-hydroxyethyl]piperazine-N′-[2-ethanesulfonic acid]). The concentration of salt and pH may need to be altered to facilitate partitioning of the active depending on the actives charge. Preferably the aqueous buffer used is 50 mM monobasic sodium phosphate, pH 8.0 or 25 mM sodium bicarbonate, pH 8.3. Examples of oils include but are not limited to oils from the following seed; rapeseed (Brassica spp), soybean (Glycine max), sunflower (Helianthus annuus), oil palm (Elaeis guineeis), olive (Olea spp.), cottonseed (Gossypium spp.), groundnut (Arachis hypogaea), coconut (Cocus nucifera), castor (Ricinus communis), safflower (Carthamus tinctorius), mustard (Brassica spp. and Sinapis alba), coriander (Coriandrum sativum), squash (Cucurbita maxima), linseed/flax (Linum usitatissimum), Brazil nut (Bertholletia excelsa), jojoba (Simmondsia chinensis), maize (Zea mays), crambe (Crambe abyssinica) and eruca (Eruca sativa). Other examples of oils include, but are not limited to, synthetic oils, mineral oil, and silicone oil. In a preferred embodiment the second solvent is safflower oil. The term “fatty acid” is used herein to describe a long hydrocarbon chain terminating in a carboxyl group. Fatty acids are the major component of lipids such as oils, fats and waxes. Examples of fatty acids include, but are not limited to, arachidic acid, arachidonic acid, beenic acid, brassidic acid, capric acid, caprylic acid, cerotic acid, cetoleic acid, erucic acid, gadoleic acid, lauric acid, lauroleic acid, lignoceric acid, linoleic acid, linolenic acid, margaric acid, mellisic/triacontanoic acid, miristoleic acid, montanic acid, myristic acid, oleic acid, palmitic acid, palmitoleic acid, steric acid, selacoleic or nervonic acid, stearic acid. The term “lipids” as used herein refer to a general group of organic substances that are insoluble in polar solvents, such as water, but readily dissolve in nonpolar organic solvents, such as chloroform, ether, benzene. Many, although not all, contain fatty acids as major structural components.

Oil Bodies

The term “oil bodies” as used herein means any discrete subcellular oil or wax storage organelle. The oil bodies may be obtained from any cell containing oil bodies or oil body-like organelles. This includes animal cells, plant cells, fungal cells, yeast cells (Leber, R. et al., 1994, Yeast 10: 1421-1428), bacterial cells (Pieper-Fürst et al., 1994, J. Bacteriol. 176: 4328-4337) and algae cells (Roessler, P. G., 1988, J. Phycol. (London) 24: 394-400). In preferred embodiments of the invention the oil bodies are obtained from a plant cell which includes cells from pollens, spores, seed and vegetative plant organs in which oil bodies or oil body-like organelles are present (Huang, 1992, Ann. Rev. Plant Physiol. 43: 177-200). More preferably, the oil body preparation of the subject invention is obtained from a plant seed. Among the plant seeds useful herein preferred are those seeds obtainable from plant species selected from the group of plant species consisting of almond (Prunus dulcis); anise (Pimpinella anisum); avocado (Persea spp.); beach nut (Fagus sylvatica); borage (also known as evening primrose) (Boragio officinalis); Brazil nut (Bertholletia excelsa); candle nut (Aleuritis tiglium); carapa (Carapa guineensis); cashew nut (Ancardium occidentale); castor (Ricinus communis); coconut (Cocus nucifera); coriander (Coriandrum sativum); cottonseed (Gossypium spp.); crambe (Crambe abyssinica); Crepis alpina; croton (Croton tiglium); Cuphea spp.; dill (Anethum gravealis); Euphorbia lagascae; Dimorphoteca pluvialis; false flax (Camolina sativa); fennel (Foeniculum vulgaris); groundnut (Arachis hypogaea); hazelnut (coryllus avellana); hemp (Cannabis sativa); honesty plant (Lunnaria annua); jojoba (Simmondsia chinensis); kapok fruit (Ceiba pentandra); kukui nut (Aleuritis moluccana); Lesquerella spp., linseed/flax (Linum usitatissimum); lupin (Lupinus spp.); macademia nut (Macademia spp.); maize (Zea mays); meadow foam (Limnanthes alba); mustard (Brassica spp. and Sinapis alba); olive (Olea spp.); oil palm (Elaeis guineeis); oiticia (Licania rigida); paw paw (Assimina triloba); pecan (Juglandaceae spp.); perilla (Perilla frutescens); physic nut (Gatropha curcas); pilinut (Canarium ovatum); pine nut (pine spp.); pistachio (Pistachia vera); pongam (Bongamin glabra); poppy seed (Papaver soniferum); rapeseed (Brassica spp.); safflower (Carthamus tinctorius); sesame seed (Sesamum indicum); soybean (Glycine max); squash (Cucurbita maxima); sal tree (Shorea rubusha); Stokes aster (Stokesia laevis); sunflower (Helianthus annuus); tukuma (Astocarya spp.); tung nut (Aleuritis cordata); vernonia (Vernonia galamensis); and mixtures thereof. Most preferably the plant seeds are from the group of plant species comprising: rapeseed (Brassica spp.), soybean (Glycine max), sunflower (Helianthus annuus), oil palm (Elaeis guineeis), cottonseed (Gossypium spp.), groundnut (Arachis hypogaea), coconut (Cocus nucifera), castor (Ricinus communis), safflower (Carthamus tinctorius), mustard (Brassica spp. and Sinapis alba), coriander (Coriandrum sativum), squash (Cucurbita maxima), linseed/flax (Linum usitatissimum), Brazil nut (Bertholletia excelsa), jojoba (Simmondsia chinensis), maize (Zea mays), crambe (Crambe abyssinica) and eruca (Eruca sativa). Most preferred for use herein are oil bodies prepared from safflower (Carthamus tinctorius).

In order to prepare oil bodies from plants, such plants are grown and allowed to set seed using agricultural cultivation practices well known to a person skilled in the art. After harvesting the seed and, if desired, removal of material such as stones or seed hulls (dehulling), by for example sieving or rinsing, and optionally drying of the seed, the seeds are subsequently processed by mechanical grinding. Preferably, a liquid phase is added prior to grinding of the seeds. This is known as wet milling. Preferably the liquid is water, although organic solvents such as ethanol may also be used. Wet milling in oil extraction processes has been reported for seeds from a variety of plant species including: mustard (Aguilar et al 1991, Journal of Texture studies 22:59-84), soybean (U.S. Pat. No. 3,971,856; Cater et al., 1974, J. Am. Oil Chem. Soc. 51:137-141), peanut (U.S. Pat. No. 4,025,658; U.S. Pat. No. 4,362,759), cottonseed (Lawhon et al., 1977, J. Am. Oil, Chem. Soc. 54:75-80) and coconut (Kumar et al., 1995, INFORM 6 (11):1217-1240). It may also be advantageous to imbibe the seeds for a time period from about fifteen minutes to about two days in a liquid phase prior to grinding. Imbibing may soften the cell walls and facilitate the grinding process. Imbibition for longer time periods may mimic the germination process and result in certain advantageous alterations in the composition of the seed constituents.

The seeds are preferably ground using a colloid mill. Besides colloid mills, other milling and grinding equipment capable of processing industrial scale quantities of seed may also be employed in the here described invention including: disk mills, colloid mills, pin mills, orbital mills, IKA mills and industrial scale homogenizers. The selection of the mill may depend on the seed throughput requirements as well as on the source of the seed that is employed. It is of critical importance that seed oil bodies remain intact during the grinding process. Therefore, any operating conditions commonly employed in oil seed processing, which tend to disrupt oil bodies are unsuitable for use in the process of the subject invention. Milling temperatures are preferably between 10° C. and 90° C. and more preferably between 25° C. and 50° C. and most preferably between 30° C. and 40° C., while the pH is preferably maintained between 2.0 and 11, more preferably between 6.0 and 9.0, and most preferably between 7.0 and 8.5.

Solid contaminants, such as seed hulls, fibrous material, undisolved carbohydrates and proteins and other insoluble contaminants are removed from the ground seed fraction. Separation of solid contaminants may be accomplished using a decantation centrifuge. Depending on the seed throughput requirements, the capacity of the decantation centrifuge may be varied by using other models of decantation centrifuges, such as 3-phase decanters. Operating conditions vary depending on the particular centrifuge which is employed and must be adjusted so that insoluble contaminating materials sediment and remain sedimented upon decantation. A partial separation of the oil body phase and liquid phase may be observed under these conditions.

Following the removal of insoluble contaminants, the oil body phase is separated from the aqueous phase. In one embodiment of the invention a tubular bowl centrifuge is employed. In a preferred embodiment a disc stack centrifuge is employed. In other embodiments, hydrocyclones, or settling of phases under natural gravitation or any other gravity based separation method may be employed. It is also possible to separate the oil body fraction from the aqueous phase employing size exclusion methods, such as filtration, for example, membrane ultrafiltration and crossflow microfiltration. A important parameter is the size of the ring dam used to operate the centrifuge. Ring dams are removable rings with a central circular opening varying, in size and regulate the separation of the aqueous phase from the oil body phase thus governing the purity of the oil body fraction that is obtained. The exact ring dam size employed depends on the type of centrifuge that is used, the type of oil seed that is used as well as on the desired final consistency of the oil body preparation. In accordance herewith in one embodiment safflower oil bodies may be obtained using an SA-7 (Westphalia) disc stack centrifuge in conjunction with a ring dam size of 73 mm. The efficiency of separation is further affected by the flow rate. In this embodiment flow rates are typically maintained between 2.0 to 7.0 l/min and temperatures are preferably maintained between 26° C. and 40° C. Depending on the model centrifuge used, flow rates and ring dam sizes can be adjusted so that an optimal separation of the oil body fraction from the aqueous phase is achieved. These adjustments will be readily apparent to a skilled artisan.

Separation of solids and separation of the aqueous phase from the oil body fraction may also be carried out concomitantly using a gravity based separation method such as 3-phase tubular bowl centrifuge or a decanter or a hydrocyclone or a size exclusion based separation method.

The compositions obtained at this stage in the process, generally are relatively crude and comprise numerous seed proteins, which includes glycosylated and non-glycosylated proteins and other contaminants such as glucosinilates or breakdown products thereof. In preferred embodiments of the present invention significant amount of seed contaminants are removed. To accomplish removal of contaminating seed material, the oil body preparation obtained upon separation from the aqueous phase is washed at least once by resuspending the oil body fraction and centrifuging the resuspended fraction. This process yields what for the purpose of this application is referred to as a washed oil body preparation. The number of washes will generally depend on the desired purity of the oil body fraction. Depending on the washing conditions that are employed, an essentially pure oil body preparation may be obtained. In such a preparation the only proteins present would be oil body proteins. In order to wash the oil body fraction, tubular bowl centrifuges or other centrifuges such hydrocyclones or disc stack centrifuges may be used. Washing of oil bodies may be performed using water, buffer systems, for example, sodium chloride in concentrations between 0.01 M and at least 2 M, 0.1 M sodium carbonate at high pH (11-12), low salt buffer, such as 50 mM Tris-HCl pH 7.5, organic solvents, detergents or any other liquid phase. In embodiments where a high purity oil body fraction is considered desirable, the washes are preferably performed at high pH (11-12). The liquid phase used for washing as well as the washing conditions, such as the pH and temperature, may be varied depending on the type of seed that is used. Washing at a number of different pH's between pH 2 and pH 11-12 may be beneficial as this will allow the step-wise removal of contaminants, in particular proteins. Washing conditions are selected such that the washing step results in the removal of a significant amount of contaminants without compromising the structural integrity of the oil bodies. In embodiments where more than one washing step is carried out, washing conditions may vary for different washing steps. SDS gel electrophoresis or other analytical techniques may conveniently be used to monitor the removal of seed proteins and other contaminants upon washing of the oil bodies. It is not necessary to remove all of the aqueous phase between washing steps and the final washed oil body preparation may be suspended in water, a buffer system, for example, 50 mM Tris-HCl pH 7.5, or any other liquid phase and if so desired the pH may be adjusted to any pH between pH 2.0 and 11, more preferably between 6.0 and 9.0 and most preferably between 7.0 and 8.5.

The process to manufacture the oil body preparation may be performed in batch operations or in a continuous flow process. Particularly when disc stack are used, a system of pumps is conveniently set up to generate a continuous flow. The pumps may be for example an air operated double diaphragm pump, hydraulic, positive displacement or peristaltic pump. In order to maintain a supply of homogenous consistency to the decantation centrifuge and to the tubular bowl centrifuge, homogenizers, such as an IKA homogenizer may be added between the separation steps. In-line homogenizers may also be added in between various centrifuges or size exclusion based separation equipment employed to wash the oil body preparations. Ring dam sizes, buffer compositions, temperature and pH may differ in each washing step from the ring dam size employed in the first separation step.

Actives

In accordance with the present invention a wide variety of biologically active ingredients may be formulated with the oil bodies of the present invention. The terms “biologically active agent”, “actives”, “active agent”, and “active ingredient” as used herein mean any agent which when administered to a living organism has a detectable biological effect including any physiological, diagnostic, prophylactic or pharmacological effect. The terms are meant to include but are not limited to any pharmaceutical, therapeutic, nutraceutical, dermatological or cosmeceutical agent. Furthermore, the terms “biological active agent”, “actives”, “active agent” and “active ingredient” as used herein refer preferably to a compound that is not soluble in an oil body, water, aqueous solution, oil, fatty acid or lipid directly, whereas the active is soluble in organic solvents. The actives may be capable of enhancing or improving the physical appearance, health, fitness or performance of the surface area of the human body, including the skin, hair, scalp, teeth and nails. Actives can be loaded to clinically significant levels, with some capable of loading in excess (20-30% dry weight of active/dry weight of oil body). The amount of active formulated will depend on the desired effect and the active that is selected. In general, the amount of active will range from 0.0001% (w/w) to about 50% (w/w). More preferably however the amount of active in the final composition will range from about 0.01% (w/w) to about 20% (w/w) and most preferably from about 0.1% (wlw) to about 10% (w/w). Depending on the chemical nature of the active, the active may become incorporated in the final formulation in a variety of ways, for example an amphipathic active may partition into the phospholipid membrane of the oil body, while a hydrophobic active may partition into the lipid core of the oil body.

The active agent is dissolved in the first solvent in an amount of first solvent that is sufficient to dissolve the active agent. The amount will vary depending of the active and solvent. The amounts can be readily determined by a person ordinarily skilled in the art. For example by referring to Standard Chemical Indices which provide information on stability, including, but not limited to, The CRC, Handbook of Chemistry and Physics or The Merck Index, Merck & Co., Inc. Budavari S (Ed.). Once the active is dissolved in the first solvent, the dissolved active is mixed with the second solvent.

Preferably the first solvent is substantially removed after mixing with the second solvent. In a specific embodiment, the first solvent is substantially removed by evaporation or substantially reduced in volume by dilution. Examples of methods to evaporate the first solvent include but are not limited to exposing the sample to either a stream of compressed air, oxygen or nitrogen. Preferably the sample is exposed to a stream of nitrogen. The term “substantially removed” as used herein means that preferably about 90 to 99.9% of the first solvent is removed, more preferably about 95 to about 99.9% of the first solvent is removed and most preferably about 99 to about 99.9% of the first solvent is removed.

The oil bodies are incubated with the solvent/active mixture to facilitate partitioning of the active into the oil bodies. The incubation is preferably performed at a temperature above 0° C. and can be performed at room temperature or at an elevated temperature. The incubation may be performed overnight or longer, for example at a temperature of 34-37° C. to ensure optimal partitioning.

Hexane extraction may be used to determine the amount of active present in the free oil (free oil is defined as oil not contained within oil bodies. Note that the intact oil bodies are largely resistant to extraction by hexane alone). (Tzen et al. (1997) J. Biochem 121: 762-768.) Once the free oil has been removed from the active/oil body fraction, an analysis of the total remaining oil may be performed using for example, hexane:isopropanol, chloroform, or chloroform:methanol solution extraction. The amount of active present in both the free oil fraction and the total oil fraction can be determined by a number of methods including, but not limited to, high performance liquid chromatography (HPLC), spectrophotometry, fluorescence, or activity assays depending on the active. By comparing the amount of active in both the free oil fraction and the total oil fraction the average amount of active incorporated into the intact oil bodies can be determined. In general, the efficiency of partitioning of the active into intact oil bodies can range from about 10 to about 99.9%. More typically however the efficiency of partitioning of the active into intact oil bodies will range from about 50 to about 99% and most typically from about 90 to about 99.

The term “hydrophobic” as used herein refers to a substance which is not readily dissolved into polar solvents such as water. In general, the greater the hydrophobicity, the greater the tendency of the substance to partition into non-polar solvents. The hydrophobic nature of a molecule may be measured by the molecules partitioning coefficient. Simply put, the partitioning coefficient is the ratio of equilibrium concentrations between two immiscible phases in contact. For example, the octanol/water partitioning coefficient (K_{OW}, P_{OW}, or P value) is the ratio of a chemical/active's concentration in the octanol (non-polar) phase to its concentration in the aqueous (polar) phase of a two-phase octanol/water system. A compound with a high P value is considered relatively hydrophobic. Since measured values range from <10⁻⁴ to >10⁺⁸ (at least 12 orders of magnitude), the logarithm (log P) is commonly used to characterize its value. The logP value can be determined experimentally, for example using reverse-phase HPLC (Yamagami and Haraguchi, 2000. Chem Pharm Bull (Tokyo) 48(12): 1973-7) or by using computer programs like the Kow Win program developed by the Syracuse Research Corporation which uses an atom/fragment contribution method.

In a preferred embodiment, the log P value of the active compound ranges from about 0 to about 8. In a more preferred embodiment, the log P value ranges from about 2 to about 7. In the most preferred embodiment, the log P value ranges from about 3 to about 7.

One particularly preferred hydrophobic active, which may be used in accordance with the present invention, is clobetasol propionate. Other synonyms and derivatives for clobetasol propionate include, but are not limited to, Clobetasol, Clobetasol 17-propionate, (11β,16β)-21-chloro-9-fluoro-11-hydroxy-16-methyl-17-(1-oxopropoxy)pregna-1,4-diene-3,20-dione, Dermoval, Dermovate, Dermoxin, Dermoxinale, Temovate and derivatives thereof. Conditions that clobetasol propionate may be used to treat include inflammatory and pruritic manifestations of moderate to severe corticosteroid-responsive dermatoses. Examples of these indications include, but are not limited to, allergic reactions, atopic dermatitis, contact dermatitis, eczema, lichen planus, lichen sclerosus, phimosis, pruritis, psoriasis, scalp dermatoses, seberrheic dermatitis, and skin irritations. P Another particularly preferred hydrophobic active, which may be used in accordance with the present invention, is diclofenac. Other synonyms and derivatives for diclofenac include, but are not limited to, 2-[(2,6-Dichlorophenyl-amino]benzeneacetic acid, [o-2,6-dichloroanilino)phenyl]-acetic acid, Voltarol, Catafram (diclofenac potassium), Vlotaren (diclofenac sodium), Vlotaren-XR, Solaraze, Allvoran, Benfofen, Dealgic, Deflamat, Delphinac, Diclomax, Miclometin, Diclophlogont, Diclo-Puren, Dicloreum, Diclo-Spondyril, Delobasan, Duravolten, Ecofenac, Effekton, Lexobene, Motifene, Neriodin, Novapirina, Primofenac, Prophenatin, Rewodina, Rhumalgan, Trabona, Tsudohmin, Valetan, Voldal, Xenid and derivatives thereto. Diclofenac is a nonsteroidal anti-inflammatory anagenic effective in treating fever, pain and inflammation in the body. Conditions that diclofenac may be used to treat include, but are not limited to, the relief of pain, tenderness, inflammation (swelling) and stiffness caused by arthritis and gout, the relief of menstrual pain and pain after surgery or childbirth, rheumatoid arthritis, osteoarthritis, ankylosing spondylitis, postoperative inflammation following cataract or corneal reactive surgery and actinic keratosis.

Still another particularly preferred hydrophobic active, which may be used in accordance with the present invention, is dithranol. Other synonyms and derivatives for dithranol include, but are not limited to, 1,8-Dihydroxy-9(10H)-anthracenone, 1,8-dihydroxyanthrone, anthralin, Anthraforte, Anthranol, Anthrascalp, Antraderm, Cignolin, Dithrocream®, Drithrocreme, Dirthro-Scalp, Micanol, Psoradrate, Prosiderm, Psorin® and derivatives thereto. Indications that dithranol may be used to treat include, but are not limited to, subacute and chronic psoriasis.

One particularly preferred hydrophobic active, which may be used in accordance with the present invention, is retinoic acid. Other synonyms and derivatives for retinoic acid include, but are not limited to, (all-E)-3,7-Dimethyl-9-(2,6,6-trimethyl-1-cyclohexen-1-yl)-2,4,6,8-nonatetraenoic acid, vitamin A acid, tretinoin, Aberel, Airol, Avita, Epi-Aberel, Eudyna, Kerlocal, Renova™, Retin-A™, retinol, Vesanoic, and derivatives thereto. Conditions that retinoic acid may be used to treat include, but are not limited to, mild to moderate acne and the treatment of sun damaged (photoaged) skin (i.e. reducing fine wrinkles, mottled hyperpigmentation and roughness associated with overexposure to the sun).

Another particularly preferred hydrophobic active, which may be used in accordance with the present invention, is lidocaine. Other synonyms and derivatives for lidocaine include, but are not limited to, 2-Diethylamino-2′,6′-acetoxylidide, 2-(Diethylamino)-N-(2,6-dimethylphenyl)acetamide, Anestacon, Cuivasal, Duncaine, EMLA(R), Gravocain, Isicaine, Leostesin, lidocaine, LIDOCAINE BASE AND LIDOCAINE HCl USP, Lidothesin, Lignocaine, Rucaina, Sylestesin, omega-Diethylamino-2′,6′-dimethylacetanilide, Xylestesin, Xylocaine, Xylocitin, Xylotox, and derivatives thereto. Conditions that lidocaine may be used to treat include, but are not limited to, ventricular arrhythmias, premature ventricular contractions (PVCs), tachycardia, and fibrillation.

Yet another particularly preferred hydrophobic active, which may be used in accordance with the present invention, is clindamycin. Other synonyms and derivatives for clindamycin include, but are not limited to, 7-Deoxy-7(S)-chlorolincomycin, 7(S)-Chloro-7-deoxylincomycin, Antirobe, Cleocin, Clindamycin, Clindatech, Dalacin, Dalacin C, Dalactine, Hydrochloride monohydrate (Dalactine), Klimicin, Klimicin C, Klimicin Methyl xamido)-1-thio-L-threo-alpha-D-galacto-octopyranoside, L-threo-alpha-D-galacto-Octopyranoside, methyl 7-chloro-6,7,8-trideoxy-6-(((1-methyl-4-propyl-2-pyrrolidinyl)carbon yl) amino)-1-thio-, (2S-trans)-Hydrochloride monohydrate, Methyl 7-chloro-6,7,8-trideoxy-6-(1-methyl-trans-4-propyl-L-2-pyrrolidinecarboxamido)-1-thio-L-threo-alpha-D-galacto-octopyranoside, Methyl 7-cloro-6,7,hio-L-threo-alpha-D-galacto-octopyranosido, Sobelin, U-21251, and derivatives thereto. Conditions that clindamycin may be used to treat include, but are not limited to, bacteroides fragilis infections in infants.

Still another particularly preferred hydrophobic active, which may be used in accordance with the present invention, is benzoyl peroxide. Other synonyms and derivatives for benzoyl peroxide include, but are not limited to, 2,3,6-TBA, Acetoxyl, incidol, loroxide, Lucidol, luperco, luperox fl, nayper b and bo, Nericur, norox bzp-250, norox bzp-c-35, Novadelox, Oxy-10, OXY-5, Oxy-5, Oxy 10, oxylite, oxy wash, PanOxyl, Peroxydex, Persadox, Persa-gel, quinolor compound, sanoxit, superox, TCBA, Theraderm, Topex, Tribac, vanoxide, Xerac, Xerac BP 10, Xerac BP 5, Acnegel, aztec bpo, Benoxyl, Benzac, Benzagel 10, Benzaknen, benzaknew, Benzoic acid, peroxide, Benzoperoxide, Benzoyl peroxide, Benzoyl peroxide, remainder water, Benzoyl Superoxide, Dibenzoyl peroxide, diphenylglyoxal peroxide, BPO, BZF-60, Cadet, cadox bs, Desanden, Debroxide, dry and clear, epi-clear, fostex, Garox, and derivatives thereto. Conditions that benzoyl peroxide may be used to treat include, but are not limited to, mild to moderate acne.

Another particularly preferred hydrophobic active, which may be used in accordance with the present invention, is cyclosporine A. Other synonyms and derivatives for cyclosporine A include, but are not limited to, antibiotic s 7481f1, Ciclosporin (Cyclosporin A, Cyclosporin A, ol 27-400, Ramihyphin A, s 7481f1, and derivatives thereto. Conditions that cyclosporine A may be used to treat include, but are not limited to, reduce the body's natural immunity in patients who receive organ (for example, kidney, liver, and heart) transplants, psoriasis and rheumatoid arthritis.

In accordance herewith in another embodiment, the anticancer drug doxorubicin (also known as Adriamycin) may be used and formulated as an oil body emulsion.

The term “amphiphilic” or “amphipathic” as used herein refers to a molecule with two distinct components that differ in their affinity for solutes and solvents. One part of the molecule has an affinity for polar solvents, such as water, and is said to be hydrophilic. A second part of the molecule has an affinity for non-polar solvents, such as hydrocarbons, and it is said to be hydrophobic. Amphipathic molecules display a distinct behaviour when interacting with water wherein the polar or hydrophilic part of the molecule “seeks” to interact with the water while the non-polar or hydrophobic part “shuns” interaction with water. The balance between the hydrophilic and lipophilic moieties in an amphipathic molecule is used as a method of classification (hydrophile-lipophile balance, HLB). The HLB values for commonly-used amphipathic molecules are readily available in the literature (eg. Handbook of Pharmaceutical Excipients, The Pharmaceutical Press. London, 1994). The HLB system was originally devised by Griffin (J. Soc. Cosmetic Chem., 1, 311, 1949). Griffin defined the HLB value of a amphipathic molecule as the mol % of the hydrophilic groups divided by 5, where a completely hydrophilic molecule (with no non-polar groups) had an HLB value of 20. This simple approach to calculating the HLB value is only applicable to polyoxyethylene ethers. Consequently, for other amphipathic molecules, HLB values have been derived from diverse properties such as water solubility, dielectric constant, interfacial tension and cloud point. Such dispersing agents preferably have a Hydrophilic-Lipophilic-Balance between 1 and 20 (HLB number, as defined in Griffin, W C, J. Soc. Cos. Chem. 1, 1949. 311: J. Soc. Cos. Chem. 5, 1954, 249). Davis et al. Proc. 2ns Int. Cong. Surface Act. Vol 1 Butterworths, 1959, London proposed a more general empirical equation that associates a constant to the different hydrophilic and hydrophobic groups:
HLB=[(n_{H}×H)−n_{L×L]+}7
Where H and L are constants assigned to hydrophilic and hydrophobic groups respectively, and n_{H} and n_{L} the number of these groups per molecule. For the purpose of the present invention, HLB is an empirical quantity, on an arbitrary scale, which is a measure of the polarity of an amphipathic molecule or mixture of amphipathic molecules. See P. Becher et al., “Nonionic Surfactant, Physical Chemistry,” Marcel Dekker, N.Y. (1987), pages 439456. Preferably, in accordance with the present invention, the HLB value of the amphipathic active ranges from about 1 to about 14, more preferably, the HLB of the active ranges from about 4 to about 10 and most preferably, the HLB value of the active ranges from about 6 to 8.

One particularly preferred amphipathic active, which may be used in accordance with the present invention, is amphotericin B. Other synonyms and derivatives for amphotericin B include, but are not limited to, amphotericin B deoxycholate, Fungizone™ and derivatives thereto. Conditions that amphotericin B may be used to treat include, but are not limited to, fungal infections.

One particularly preferred amphipathic active, which may be used in accordance with the present invention, is phosphatidyl choline. Other synonyms for phosphatidyl choline include, but are not limited to, lecithin and derivatives thereto. Phosphatidyl choline is a membrane phospholipid. Phosphatidyl choline is found in many skin care products and has been used in cosmetic surgery (i.e. injected into fat pads under eyes to minimize puffiness).

One particularly preferred amphipathic active, which may be used in accordance with the present invention, is tetracaine. Other synonyms and derivatives for tetracaine include, but are not limited to, amethocaine, 2-dimethylaminoethyl, 4-(Butylamino)benzoic acid 2-(dimethylamino)ethyl ester monohydrochloride, 4-(Butylamino)benzoic acid, Anethaine, Butethanol, Tonexol, 4-(butylamino)-Benzoic acid, 2-(dimethylamino)ethyl ester, dicain, Decicain, Pontocaine, and derivatives thereto. Tetracine is an effective local anesthetic for topical applications. Examples of these topical applications include, but are not limited to, anesthesia prior to venepuncture or venous cannulation, and minor eye operations.

Another particularly preferred amphipathic active, which may be used in accordance with the present invention, is actinomycin. Other synonyms and derivatives for actinomycin include, but are not limited to, 3H-Phenoxazine-1,9-dicarboxamide, 2-amino-N,N′-bis [hexadecahydro-2,5,9-trimethyl-6,13-bis(1-methylethyl)-1,4,7,11,14-pentaoxo-1H-pyrrolo[2,1-i] [1,4,7,10,13]oxatetraazacyclohexadecin-10-yl]-4,6-dimethyl-3-oxo-, ACT, hbf 386, Lyovac cosmegen, Meractinomycin, Oncostatin K, X 97, Actactinomycin A IV, Actinomycin 7, Actinomycin AIV, Actinomycindioic D acid, dilactone, Actinomycin C1, actinomycin cl, Actinomycin D, (−)-actinomycin d, actinomycin i, Actinomycin I1, Actinomycin IV, Actinomycin-[threo-val-pro-sar-meval], Actinomycin X 1, actinomycin x i, actinomyein-theo-val-pro-sar-meval, ACTO-D, AD, Dilactone actinomycindioic D acid, Dilactone actinomycin D acid, C1, Cosmegen, Dactinomycin, Dactinomycin D, dactinomyein d, and derivatives thereto. Conditions that actinomomycin D may be used to treat include, but are not limited to, cancer and it is used as an antibiotic.

Further actives contemplated for use in the compositions described herein include the following categories and examples of actives and alternative forms of these actives such as alternative salt forms, free acid forms, free base forms, and hydrates:
analgesics/antipyretics (e.g., aspirin, acetaminophen, ibuprofen, naproxen sodium, buprenorphine, propoxyphene hydrochloride, propoxyphene napsylate, meperidine hydrochloride, hydromorphone hydrochloride, morphine, oxycodone, codeine, dihydrocodeine bitartrate, pentazocine, hydrocodone bitartrate, levorphanol, diflunisal, trolamine salicylate, nalbuphine hydrochloride, mefenamic acid, butorphanol, choline salicylate, butalbital, phenyltoloxamine citrate, diphenhydramine citrate, methotrimeprazine, cinnamedrine hydrochloride, and meprobamate);
antiasthamatics (e.g., ketotifen and traxanox);
antibiotics (e.g., neomycin, streptomycin, chloramphenicol, cephalosporin, ampicillin, penicillin, tetracycline, and ciprofloxacin);
antidepressants (e.g., nefopam, oxypertine, doxepin, amoxapine, trazodone, amitriptyline, maprotiline, phenelzine, desipramine, nortriptyline, tranylcypromine, fluoxetine, doxepin, imipramine, imipramine pamoate, isocarboxazid, trimipramine, and protriptyline);
antidiabetics (e.g., biguanides and sulfonylurea derivatives);
antifungal agents (e.g., griseofulvin, ketoconazole, itraconizole, amphotericin B, nystatin, and candicidin);
antihypertensive agents (e.g., propanolol, propafenone, oxyprenolol, nifedipine, reserpine, trimethaphan, phenoxybenzamine, pargyline hydrochloride, deserpidine, diazoxide, guanethidine monosulfate, minoxidil, rescinnamine, sodium nitroprusside, rauwolfia serpentina, alseroxylon, and phentolamine);
anti-inflammatories (e.g., (non-steroidal) indomethacin, ketoprofen, flurbiprofen, naproxen, ibuprofen, ramifenazone, piroxicam, biphenylcarboxylic acid derivatives, acetominaphen, (steroidal) hydrocortisone, cortisone, dexamethasone, fluazacort, celecoxib, rofecoxib, hydrocortisone, prednisolone, and prednisone);
antitneoplastics (e.g., cyclophosphamide, actinomycin, bleomycin, daunorubicin, doxorubicin, epirubicin, mitomycin, methotrexate, fluorouracil, carboplatin, carmustine (BCNU), methyl-CCNU, cisplatin, etoposide, camptothecin and derivatives thereof, phenesterine, paclitaxel and derivatives thereof, docetaxel and derivatives thereof, vinblastine, vincristine, tamoxifen, and piposulfan);
antianxiety agents (e.g., lorazepam, buspirone, prazepam, chlordiazepoxide, oxazepam, clorazepate dipotassium, diazepam, hydroxyzine pamoate, hydroxyzine hydrochloride, alprazolam, droperidol, halazepam, chlormezanone, and dantrolene);
immunosuppressive agents (e.g., cyclosporine, azathioprine, mizoribine, and FK506 (tacrolimus));
antimigraine agents (e.g., ergotamine, propanolol, isometheptene mucate, and dichloralphenazone);
sedatives/hypnotics (e.g., barbiturates such as pentobarbital, pentobarbital, and secobarbital; and benzodiazapines such as flurazepam hydrochloride, triazolam, and midazolam);
antianginal agents (e.g., beta-adrenergic blockers; calcium channel blockers such as nifedipine, and diltiazem; and nitrates such as nitroglycerin, isosorbide dinitrate, pentaerythritol tetranitrate, and erythrityl tetranitrate);
antipsychotic agents (e.g., haloperidol, loxapine succinate, loxapine hydrochloride, thioridazine, thioridazine hydrochloride, thiothixene, fluphenazine, fluphenazine decanoate, fluphenazine enanthate, trifluoperazine, chlorpromazine, perphenazine, lithium citrate, and prochlorperazine);
antimanic agents (e.g., lithium carbonate);
antiarrhythmics (e.g., bretylium tosylate, esmolol, verapamil, amiodarone, encainide, digoxin, digitoxin, mexiletine, disopyramide phosphate, procainamide, quinidine sulfate, quinidine gluconate, quinidine polygalacturonate, flecainide acetate, tocainide, and lidocaine);
antiarthritic agents (e.g., phenylbutazone, sulindac, penicillamine, salsalate, piroxicam, azathioprine, indomethacin, meclofenamate, gold sodium thiomalate, ketoprofen, auranofin, aurothioglucose, and tolmetin sodium);
antigout apents (e.g., colchicine, and allopurinol);
anticoagulants (e.g., heparin, heparin sodium, and warfarin sodium);
thrombolytic agents (e.g., urokinase, streptokinase, and alteplase);
antifibrinolytic agents (e.g., aminocaproic acid);
hemorheologic agents (e.g., pentoxifylline):
antiplatelet agents (e.g., aspirin);
anticonvulsants (e.g., valproic acid, divalproex sodium, phenytoin, phenytoin sodium, clonazepam, primidone, phenobarbitol, carbamazepine, amobarbital sodium, methsuximide, metharbital, mephobarbital, mephenytoin, phensuximide, paramethadione, ethotoin, phenacemide, secobarbitol sodium, clorazepate dipotassium, and trimethadione);
antiparkinson agents (e.g., ethosuximide);
antihistamines/antipruritics (e.g., hydroxyzine, diphenhydramine, chlorpheniramine, brompheniramine maleate, cyproheptadine hydrochloride, terfenadine, clemastine fumarate, triprolidine, carbinoxamine, diphenylpyraline, phenindamine, azatadine, tripelennamine, dexchlorpheniramine maleate, methdilazine, and);
agents useful for calcium regulation (e.g., calcitonin, and parathyroid hormone);
antibacterial agents (e.g., amikacin sulfate, aztreonam, chloramphenicol, chloramphenicol palmitate, ciprofloxacin, clindamycin, clindamycin palmitate, clindamycin phosphate, metronidazole, metronidazole hydrochloride, gentamicin sulfate, lincomycin hydrochloride, tobramycin sulfate, vancomycin hydrochloride, polymyxin B sulfate, colistimethate sodium, and colistin sulfate);
antiviral agents (e.g., interferon alpha, beta or gamma, zidovudine, amantadine hydrochloride, ribavirin, and acyclovir);
antimicrobials (e.g., cephalosporins such as cefazolin sodium, cephradine, cefaclor, cephapirin sodium, ceftizoxime sodium, cefoperazone sodium, cefotetan disodium, cefuroxime e azotil, cefotaxime sodium, cefadroxil monohydrate, cephalexin, cephalothin sodium, cephalexin hydrochloride monohydrate, cefamandole nafate, cefoxitin sodium, cefonicid sodium, ceforanide, ceftriaxone sodium, ceftazidime, cefadroxil, cephradine, and cefuroxime sodium; penicillins such as ampicillin, amoxicillin, penicillin G benzathine, cyclacillin, ampicillin sodium, penicillin G potassium, penicillin V potassium, piperacillin sodium, oxacillin sodium, bacampicillin hydrochloride. cloxacillin sodium, ticarcillin disodium, azlocillin sodium, carbenicillin indanyl sodium, penicillin G procaine, methicillin sodium, and nafcillin sodium; erythromycins such as erythromycin ethylsuccinate, erythromycin, erythromycin estolate, erythromycin lactobionate, erythromycin stearate, and erythromycin ethylsuccinate; and tetracyclines such as tetracycline hydrochloride, doxycycline hyclate, and minocycline hydrochloride, azithromycin, clarithromycin, triclosan, tolnafiate, chlorhexidine, benzoyl peroxide)
anti-infectives (e.g., GM-CSF);
bronchodilators (e.g., sympathomimetics such as epinephrine hydrochloride, metaproterenol sulfate, terbutaline sulfate, isoetharine, isoetharine mesylate, isoetharine hydrochloride, albuterol sulfate, albuterol, bitolterolmesylate, isoproterenol hydrochloride, terbutaline sulfate, epinephrine bitartrate, metaproterenol sulfate, epinephrine, and epinephrine bitartrate; anticholinergic agents such as ipratropium bromide; xanthines such as aminophylline, dyphylline, metaproterenol sulfate, and aminophylline; mast cell stabilizers such as cromolyn sodium; inhalant corticosteroids such as beclomethasone dipropionate (BDP), and beclomethasone dipropionate monohydrate; salbutamol, ipratropium bromide; budesonide; ketotifen; salmeterol; xinafoate; terbutaline sulfate; triamcinolone; theophylline; nedocromil sodium; metaproterenol sulfate; albuterol; flunisolide; fluticasone proprionate
steroidal compounds and hormones (e.g., androgens such as danazol, testosterone cypionate, fluoxymesterone, ethyltestosterone, testosterone enathate, methyltestosterone, fluoxymesterone, and testosterone cypionate; estrogens such as estradiol, estropipate, and conjugated estrogens; progestins such as methoxyprogesterone acetate, and norethindrone acetate; corticosteroids such as triamcinolone, betamethasone, betamethasone sodium phosphate, dexamethasone, dexamethasone sodium phosphate, dexamethasone acetate prednisone, methylprednisolone acetate suspension, triamcinolone acetonide, methylprednisolone, prednisolone sodium phosphate, methylprednisolone sodium succinate, hydrocortisone sodium succinate, triamcinolone hexacetonide, hydrocortisone, hydrocortisone cypionate, prednisolone, fludrocortisone acetate, paramethasone acetate, prednisolone tebutate, prednisolone acetate, prednisolone sodium phosphate, and hydrocortisone sodium succinate; and thyroid hormones such as levothyroxine sodium);
hypoglycemic agents (e.g., human insulin, purified beef insulin, purified pork insulin, glyburide, chlorpropamide, glipizide, tolbutamide, and tolazamide);
hypolipidemic agents (e.g., clofibrate, dextrothyroxine sodium, probucol, pravastitin, atorvastatin, lovastatin, and niacin);
proteins (e.g., DNase, alginase, superoxide dismutase, and lipase); nucleic acids (e.g., sense or anti-sense nucleic acids encoding any therapeutically useful protein, including any of the proteins described herein);
agents useful for erythropoiesis stimulation (e.g., erythropoietin);
antiulcer/antireflux agents (e.g., famotidine, cimetidine, and ranitidine hydrochloride);
antinauseants/antiemetics (e.g., meclizine hydrochloride, nabilone, prochlorperazine, dimenhydrinate, promethazine hydrochloride, thiethylperazine, and scopolamine);
oil-soluble vitamins (e.g., vitamins A, D, E, K, and the like); as well as other drugs such as mitotane, halonitrosoureas, anthrocyclines, and ellipticine.
sunscreen actives (e.g. para-amino benzoic acid (PABA), octyl salicylate, octyl methoxy cinnamate (Parasol MCX); titanium dioxide)
anti-wrinkle and anti-aging actives (e.g. retinoic acid)
whitening and bleaching actives (e.g. ascorbyl parmitate and licorice root)
anti-acne actives (e.g. hydrocortisone and benzoyl peroxide)
vitamin actives (e.g. Vitamin A and derivatives including retinoic acid, retinyl aldehyde, retin A, retinyl palmitate, adapalene, and beta-carotene; vitamin D including calcipotriene (a vitamin D3 analog); vitamin E including its individual constituents alpha-, beta-, gamma-, delta-tocopherol and cotrienols and mixtures thereof and vitamin E derivatives including vitamin E palmitate, vitamin E linolate and vitamin E acetate; vitamin K and derivatives.)
lipids (e.g. inter alia triacyl glycerides; fatty acids such as gamma-linolenic acid; waxes; cholesterol; sphingolipids; ceramides; phospholipids and mixtures thereof.
pigments (e.g. titanium dioxide, zinc oxide, zirconium dioxide, methozsalen, trioxsalen, carotenoids (alpha carotene, beta carotene, lutein, lycopene), chlorophylls (a and b), xanthophylls.
antioxidants (e.g. Vitamin E and vitamin E mimetics, alpha-lipoic acid, coenzyme Q (CoQ, Q, Ubiquinone), Vitamin A, Carotenes, lycopene, lipoic acid, melatonin, some polyphenols, some flavonoids)
antifungals (e.g. rilopirox, lanoconazole, benzylamine derivatives, imadozole, amphotericin B)
fragrances (e.g. acetanisole, acetophenone, acetyl cedrene, methyl nonyl acetaldehyde, heliotropin, sandella, methoxycitranellal, hydroxycitranellal, geraniol, benzaldehyde, linalool, p-tertiairy butyl cyclohexyl acetate, cinnamyl acetate, 1-menthol, vanillin, sandlewood oil, angelic root oil, bergamont oil, buchu leaf oil, cassia oil, chamomile oil, lemon oil, lavender oil, Ylang Ylang oil)
insect repellants (e.g. N,N-Diethyl-3 methyl benzamide, N,N-diethyl-m toluamide (DEET), dimethylphthalate (DMP), oils of citronella or eucalyptus, pyrethrines, pyrethroids)
   A description of these and other classes of useful actives and a listing of species within each class can be found in Martindale, The Extra Pharmacopoeia, 30th Ed. (The Pharmaceutical Press, London 1993), the disclosure of which is incorporated herein by reference in its entirety.

The following non-limiting examples are illustrative of the present invention:

### EXAMPLES

### Example 1

Obtaining a Washed Oil Body Preparation from Safflower.

This example describes the recovery of the oil body fraction from safflower. The resulting preparation contains intact washed oil bodies.

Seed decontamination. A total of 45 kg of dry safflower (Carthamus tinctorius) seed was washed twice using approximate 120 L of 65° C. tap water and once using approximately 120 L of about 15° C. tap water. The washing was carried out in a barrel with screen mesh to separate the waste water.

Grinding of seeds. The washed seeds were poured through the hopper of a colloid mill (Colloid Mill, MZ-130 (Fryma); capacity: 350 kg/hr), which was equipped with a MZ-120 crosswise toothed rotor/stator grinding set and top loading hopper, while approximately 100 L of 25 mM NaH₂PO₄ buffer of pH7.0 was supplied through an externally connected hose prior to milling. Operation of the mill was at a gap setting of 1 R, chosen to achieve a particle size less than 100 micron at 18° C. and 30° C. All 45 kg of seeds were ground in 10 minutes.

Homogenization and Removal of solids. The resulting slurry was pumped into a knife in-line homogenizer (Dispax Reactor® DR 3-6/A, IKA® Works, Inc.) at a speed about 7 L/min. The output slurry was directly fed into a decantation centrifuge (NX-314B-31, Alfa-Laval) after bringing the centrifuge up to an operating speed of 3250 rpm. In 25 minutes approximately 160 kg of seed ground slurry was decanted. A Watson-Marlow (Model 704) peristaltic pump was used for slurry transfer in this step.

Oil body separation. Separation of the oil body fraction was achieved using a disc-stack centrifuge separator (SA 7, Westfalia) equipped with a three phase separating and self-cleaning bowl and removable ring dam series; maximum capacity:83 L/min; ringdam: 69 mm. Operating speed was at ˜8520 rpm. A Watson-Marlow (Model 704) peristaltic pump was used to pump the decanted liquid phase (DL) into the centrifuge after bringing it up to operating speed. This results in separation of the decanted liquid phase into a heavy phase (HP1) comprising water and soluble seed proteins and a light phase (LP1) comprising oil bodies. The oil body fraction, which was obtained after one pass through the centrifuge, is referred to as an unwashed oil body preparation. This unwashed oil body fraction was then passed through a static inline mixer, mixing with, 25 mM NaH₂PO₄ (pH 7) buffer (35° C., 4 L/min) into a second disc-stack centrifuge separator (SA 7, Westfalia); maximum capacity:83 L/min; ringdam: 73 mm. Operating speed was at ˜8520 rpm. The separated light phase (LP2) comprising oilbodies was then passed through another static inline mixer mixing with pH8, 50 mM NaH₂PO₄ buffer (35° C., 4 L/min) into the third disc-stack centrifuge separator (SA 7, Westfalia); maximum capacity:83 L/min; ringdam: 75 mm. Operating speed was at ˜8520 rpm. The entire procedure was carried out at room temperature. The preparations obtained following the second separation are all referred to as the washed oil body preparation. Following three washes much of the contaminating soluble seed protein was removed. If the oil bodies are used in a cosmetic formulation, then 0.1% Neolone 950 and 0.1% glycacil L may be added as preservatives.

### Example 2

Partitioning of Clobetasol Propionate into Washed Safflower Oil Bodies.

Washed safflower oil bodies were prepared as described in example 1. Oil bodies were preserved with 0.1 % Neolone 950 and 0.1 % glycacil L. Clobetasol propionate (CP) (Supplier-Sigma) was weighed (12-30 mg) into a clean and dry 16×100 mm screw-cap Pyrex test tube and mixed with 300 μl of isopropanol then 200 mg of safflower oil. The combined sample was vortexed and subsequently incubated at 34° C. for 20 minutes. After incubation, the sample was re-vortexed then dried under nitrogen for 20 minutes to remove the isopropanol. One ml of washed high dry weight oil bodies were added to the CP/safflower oil mixture at room temperature, centrifuged briefly to pellet contents to allow for thorough mixing, vortexed and further incubated at 34-42° C. overnight in an air tight tube to allow for incorporation of the CP and safflower oil into the oil bodies. A hexane extraction was used to determine the amount of CP present in the free oil (free oil is defined as oil not contained within oil bodies. Note that the intact oil bodies are largely resistant to extraction by hexane alone but all hexane extractions of loaded oil bodies must be corrected for damage done to intact oil bodies by the hexane through corrections using free oil values obtained from unloaded oilbodies). Three ml of hexane are added to the tubes and the tubes are shaken to mix 32 times. The samples are centrifuged in a swinging bucket clinical centrifuge at 3220×g for one minute to separate hexane from the oil body-aqueous phase. Note that the free oil will remain in the hexane layer (top). The hexane layer is removed to another tube and the hexane extraction is repeated on the remaining CP/oil body mixture. Once the majority of the solvent from the second extraction is added to the tube containing the first extract, the hexane containing tube is transferred to a heating block. The hexane was evaporated by subjecting the tubes to a gentle stream of high purity N₂ gas while heating the block to 40-45° C. for at least 1.5 hours. Once the free oil was removed from the CP/oil body fraction, an analysis of the total remaining oil was performed. The remaining total oil in intact oil bodies was determined by adding 4 ml of a 3:2 hexane:isopropanol solution (HIP) and shaking to mix vigorously until all of the oil was dissolved in the HIP solvent (about 10-20 seconds). This was followed by the addition of 2.5 ml of 6.67% Na₂SO₄ (w/v) to the tube and the tube was shaken for another 10 seconds. Phase separation is facilitated by centrifugation for 2 minutes at 3220×g in a swinging bucket clinical centrifuge. The organic or upper phase is removed to a second test tube using a Pasteur pipette while avoiding the transfer of the aqueous phase. Three ml of a 7:2 HIP solution was added to the original tube containing the aqueous phase and the tube shaken for 10 seconds. The tube was centrifuged for 2 minutes at 3220×g and the upper phase is combined with the organic phase retrieved in the first HIP extraction. The 7:2 HIP extraction step was repeated. The solvent was evaporated from the lipid extract by subjecting the tube containing the combined organic phases to a gentle stream of compressed N₂ gas while heating (40-45° C.) in a dry block heater. The tube is weighed after one hour and then every 15 minutes after that When two successive weights are the same (±0.0001 g), then it is assumed that all volatile components have evaporated and that only extracted lipids remain. The amount of CP present in both the free oil fraction and the total oil fraction was determine by high performance liquid chromatography (HPLC) at a wave-length of 240 nm and then compared to an HPLC standard curve prepared with known amounts of CP. By comparing the amount of CP in both the free oil fraction and the total oil fraction it was determined that an average of 94.7% of the clobetasol propionate added was incorporated into the intact oil bodies. The level of clobetasol propionate incorporated into the oil bodies as a percentage of dry weight is 0.316%. When loading larger volumes of oil bodies, the Cito-unguator lab mixer (Gako Konietzko) was found to be particularly efficient at mixing the oil with the oil bodies thus promoting efficient loading.

### Example 3

Partitioning of Retinoic Acid into Washed Safflower Oil Bodies.

Washed safflower oil bodies were prepared as described in example 1. Oil bodies were preserved with 0.1% Neolone 950 and 0.1% glycacil L. Retinoic acid (RA) (Supplier-Sigma) was weighed (1-8 mg) into a clean and dry 16×100 mm screw-cap Pyrex test tube and mixed with 3 ml of isopropanol. Safflower oil is added so that there is not more than 5 mg of RA per gram of safflower oil. The combined sample was vortexed, placed in a 40-45° C. heating block, then dried under a steady stream of nitrogen until the isopropanol is removed (about 0.5-1 hour). Next 4 to 5 ml of washed high dry weight oil bodies is added to the RA/safflower oil mixture per gram of safflower oil used to solubilize the RA. Note that the RA/safflower oil mixture was kept in the heating block until immediately before the addition of the oil bodies. This oil body mixture is centrifuged briefly to pellet the contents which allows for thorough mixing of the contents, vortexed and further incubated at 34-37° C. overnight in an air tight tube to allow for incorporation of the RA and safflower oil into the oil bodies. A hexane extraction was used to determine the amount of free oil to determine the amount of un-incorporated RA still solubilized in free oil (free oil is defined as oil not contained within oil bodies. Note that that intact oil bodies are largely resistant to extraction by hexane alone but all hexane extractions of loaded oil bodies must be corrected for damage done to intact oil bodies by the hexane through corrections using free oil values obtained from unloaded oilbodies). After 3 ml of hexane are added to the tubes the tubes are shaken to mix 32 times. The samples are centrifuged in a swinging bucket clinical centrifuge at 3220×g for one minute to separate hexane form the oil body-aqueous phase. Note that the free oil will remain in the hexane layer (top). The hexane layer is removed to another tube and the hexane extraction is repeated on the remaining RA/oil body mixture. Once the majority of the solvent from the second extraction is added to the tube containing the first extract, the hexane containing tube is transferred to a heating block. The hexane was evaporated by subjecting the tubes to a gentle stream of high purity N₂ gas while heating the block to 40-45° C. for at least 1.5 hours. Once the free oil was removed from the RA/oil body fraction, an analysis of the total remaining oil was performed. The remaining total oil in intact oil bodies was determined by adding 4 ml of a 3:2 hexane:isopropanol solution (HIP) and shaking to mix vigorously until all of the oil was dissolved in the HIP solvent (about 10-20 seconds). This was followed by the addition of 2.5 ml of 6.67% Na₂SO₄ (w/v) to the tube and the tube was shaken for another 10 seconds. Phase separation is facilitated by centrifugation for 2 minutes at 3220×g in a swinging bucket clinical centrifuge. The organic or upper phase is removed to a second test tube using a Pasteur pipette while avoiding the transfer of the aqueous phase. Three ml of a 7:2 HIP solution was added to the original tube containing the aqueous phase and the tube shaken for 10 seconds. The tube was centrifuged for 2 minutes at 3220×g and the upper phase is combined with the organic phase retrieved in the first HIP extraction. The 7:2 HIP extraction step was repeated. The solvent was evaporated from the lipid extract by subjecting the tube containing the combined organic phases to a gentle stream of compressed N₂ gas while heating (40-45° C.) in a dry block heater. The tube is weighed after one hour and then every 15 minutes after that. When two successive weights are the same (±0.0001 g), then it is assumed that all volatile components have evaporated and that only extracted lipids remain. The amount of RA present in both the free oil fraction and the total oil fraction was calculated by measuring the absorbance using a spectrometer at a wave length of 380 nm and then compared to a standard curve prepared with known amounts of RA. By comparing the amount of RA recovered from the total oil fraction to what was added to the oil bodies, it was determined that an average of 94.72% of the RA added was incorporated into the intact oil bodies. The level of RA incorporated into the oil bodies as a percentage of dry weight is 0.195%. When loading larger volumes of oil bodies, the Cito-unguator lab mixer (Gako Konietzko) was found to be particularly efficient at mixing the oil with the oil bodies thus promoting efficient loading.

### Example 4

Partitioning of Dithranol into Washed Safflower Oil Bodies.

Washed safflower oil bodies were prepared as described in example 1. Oil bodies were preserved with 0.1% Neolone 950 and 0.1% glycacil L. Dithranol (Supplier-Spectrum) was weighed (1-30 mg) into a clean and dry 16×100 mm screw-cap Pyrex test tube and mixed with 500 μl of chloroform. Safflower oil is added so that there is not more than 9 mg of dithranol per gram of safflower oil. The combined sample was vortexed, place in a 40-45° C. heating block, then dried under a steady stream of nitrogen until the chloroform is removed (about 1-2 hours with occasional remixing of the mixture). Next 4 to 5 ml of washed high dry weight oil bodies containing 0.2% L-ascorbic acid (Supplier-Sigma) is added at room temperature to the dithranol/safflower oil mixture per gram of safflower oil used to solubilize the dithranol. This oil body mixture is centrifuged briefly to pellet the contents to allow for thorough mixing, vortexed and further incubated at 34-37° C. overnight in an air tight tube to allow for incorporation of the dithranol and safflower oil into the oil bodies. The oil bodies are washed once with an equal volume of 50 mM phosphate, pH 8.0, containing 0.2% L-ascorbic acid (Note: unincorporated dithranol can pellet in the wash. Dithranol is not soluble in hexane so hexane extractions will not remove the unincorporated dithranol.) and the oil bodies are removed from the top and placed in a fresh tube. A hexane extraction was used to determine the amount of free oil indicating the efficiency of loading of the oil carrier (free oil is defined as oil not contained within oil bodies. Note that the intact oil bodies are largely resistant to extraction by hexane alone but all hexane extractions of loaded oil bodies must be corrected for damage done to intact oil bodies by the hexane through corrections using free oil values obtained from unloaded oilbodies). Then 3 ml of hexane is added to the tubes and the tubes are shaken 32 times to mix. The samples are centrifuged in a swinging bucket clinical centrifuge at 3220×g for one minute to separate hexane from the oil body-aqueous phase. Note that the free oil will remain in the hexane layer (top). The hexane layer is removed to another tube and the hexane extraction is repeated on the remaining dithranol/oil body mixture. Once the majority of the solvent from the second extraction is added to the tube containing the first extract, the hexane containing tube is transferred to a heating block. The hexane was evaporated by subjecting the tubes to a gentle stream of high purity N₂ gas while heating the block to 40-45° C. for at least 1.5 hours. Once the free oil was removed from the dithranol/oil body fraction, an analysis of the total remaining oil was performed. The remaining total oil in intact oil bodies was determined by adding 3 ml of chloroform and shaking vigorously to mix, until all of the oil was dissolved in the solvent (about 10-20 seconds). Phase separation is facilitated by centrifugation for 1 minute at 3220×g in a swinging bucket clinical centrifuge. The organic or lower phase is removed to a second test tube using a Pasteur pipette, while avoiding the transfer of the aqueous phase, and 3 ml of chloroform was added to the original tube containing the aqueous phase and the tube shaken for 10 seconds. The tube was centrifuged for 1 minute at 3220×g and the lower phase is combined with the organic phase retrieved in the first chloroform extraction. To the original tube containing the aqueous phase 3 ml of a 7:2 hexane:isopropanol solution (HIP) was added and the tube was shaken for 10 seconds. The tube was then centrifuged for 2 minutes at 3220×g and the upper phase is combined with the lower chloroform phase obtained in the first 2 steps. The 7:2 HIP extraction step was repeated. The solvent was evaporated from the lipid extract by subjecting the tube containing the combined organic phases to a gentle stream of compressed N₂ gas while heating (40-45° C.) in a dry block heater. The tube is weighed after one hour and then every 15 minutes after that. When two successive weights are the same (±0.0001 g), it is assumed that all volatile components have evaporated and that only extracted lipids remain. The amount of dithranol present in both the free oil fraction and the total oil fraction was calculated by measuring the absorbance using a spectrometer at a wave length of 376 nm and then compared to a standard curve prepared with known amounts of dithranol. By comparing the amount of dithranol recovered from the total oil fraction to what was added to the oil bodies, it was determined that an average of 97.3% of the dithranol added was incorporated into the intact oil bodies. The level of dithranol incorporated into the oil bodies as a percentage of dry weight is 0.253%. When loading larger volumes of oil bodies, the Cito-unguator lab mixer (Gako Konietzko) was found to be particularly efficient at mixing the oil with the oil bodies thus promoting efficient loading.

### Example 5

Partitioning of Diclofenac into Washed Safflower Oil Bodies.

Washed safflower oil bodies were prepared as described in example 1. Oil bodies were preserved with 0.1% Neolone 950 and 0.1% glycacil L. Diclofenac (Supplier-Sigma) was weighed (1-300 mg) into a clean and dry 16×100 mm screw-cap Pyrex test tube and mixed with 10 ml of ethanol and 3 volumes of phosphate buffer (50 mM monobasic sodium phosphate, pH 8.0, with 0.1% Neolone 950) was added to the ethanol/diclofenac mix. One g of oil bodies is added to the buffered ethanol mix at room temperature and the sample is mixed well and incubated at 34-37° C. overnight in an air tight tube to allow for incorporation of the diclofenac into the oil bodies. The oil bodies are centrifuged for 10 minutes at 3220×g to separate them from the buffer containing the ethanol and presumably the unincorporated diclofenac. The buffer portion is removed and the oil bodies are washed twice in 2 volumes of 50 mM phosphate, pH 8.0 containing 0.1% Neolone 950. A chloroform::methanol (2:1) extraction was used to determine the amount of diclofenac contained within the oil extracted from the oil bodies. Then 3 ml of chloroform::methanol is added to the tubes and the tubes are shaken vigorously to mix. The samples are centrifuged in a swinging bucket clinical centrifuge at 3220×g for one minute to separate the solvent from the oil body-aqueous phase. The solvent layer is removed to another tube and the extraction is repeated twice more on the remaining diclofenac/oil body mixture. The second and third extractions were added to the tube containing the first extract and the tube containing the extracts is transferred to a heating block. The solvents were evaporated by subjecting the tubes to a gentle stream of high purity N₂ gas while heating the block to 40-45° C. for at least 1.5 hours. The tube is weighed after one hour and then every 15 minutes after that. When two successive weights are the same (±0.0001 g), it is assumed that all volatile components have evaporated and that only extracted lipids remain. The amount of diclofenac present in the total oil fraction was calculated by measuring the absorbance using a spectrometer at a wave-length of 320 nm and then compared to a standard curve prepared with known amounts of diclofenac. By comparing the amount of diclofenac recovered from the total oil fraction to what was added to the oil bodies, it was determined that an average of 43.9% of the diclofenac added was incorporated into the intact oil bodies. The level of diclofenac incorporated into the oil bodies as a percentage of dry weight is 1.36%.

### Example 6

Partitioning of Tetracaine into Washed Safflower Oil Bodies.

Washed safflower oil bodies were prepared as described in example 1. Oil bodies were preserved with 0.1% Neolone 950 and 0.1% glycacil L. Tetracaine free base (Supplier-Sigma) was weighed (1-200 mg) into a clean and dry 16×100 mm screw-cap Pyrex test tube and mixed with 1 ml of isopropanol then 1 gram of safflower oil. Next 3-5 g of high dry weight oil bodies are added at room temperature to the tetracaine/oil mix and the sample is mixed well and incubated at 34-37° C. overnight in an air tight tube to allow for incorporation of the tetracaine into the oil bodies. A hexane extraction was used to determine the amount of free oil to determine the amount of un-incorporated tetracaine still solubilized in free oil (free oil is defined as oil not contained within oil bodies. Note that that intact oil bodies are largely resistant to extraction by hexane alone but all hexane extractions of loaded oil bodies must be corrected for damage done to intact oil bodies by the hexane through corrections using free oil values obtained from unloaded oilbodies). After 3 ml of hexane are added to the tubes the tubes are shaken to mix 32 times. The samples are centrifuged in a swinging bucket clinical centrifuge at 3220×g for one minute to separate hexane form the oil body-aqueous phase. Note that the free oil will remain in the hexane layer (top). The hexane layer is removed to another tube and the hexane extraction is repeated on the remaining tetracaine/oil body mixture. Once the majority of the solvent from the second extraction is added to the tube containing the first extract, the hexane containing tube is transferred to a heating block. The hexane was evaporated by subjecting the tubes to a gentle stream of high purity N₂ gas while heating the block to 40-45° C. for at least 1.5 hours. Once the free oil was removed from the tetracaine/oil body fraction, an analysis of the total remaining oil was performed. The remaining total oil in intact oil bodies was determined by adding 4 ml of a 3:2 hexane:isopropanol solution (HIP) and shaking to mix vigorously until all of the oil was dissolved in the HIP solvent (about 10-20 seconds). This was followed by the addition of 2.5 ml of 6.67% Na₂SO₄ (w/v) to the tube and the tube was shaken for another 10 seconds. Phase separation is facilitated by centrifugation for 2 minutes at 3220×g in a swinging bucket clinical centrifuge. The organic or upper phase is removed to a second test tube using a Pasteur pipette while avoiding the transfer of the aqueous phase. Three ml of a 7:2 HIP solution was added to the original tube containing the aqueous phase and the tube shaken for 10 seconds. The tube was centrifuged for 2 minutes at 3220×g and the upper phase is combined with the organic phase retrieved in the first HIP extraction. The 7:2 HIP extraction step was repeated. The solvents were evaporated by subjecting the tubes to a gentle stream of high purity N₂ gas while heating the block to 40-45° C. for at least 1.5 hours. The tube is weighed after one hour and then every 15 minutes after that. When two successive weights are the same (±0.0001 g), it is assumed that all volatile components have evaporated and that only extracted lipids remain. The amount of tetracaine present in the total oil fraction was calculated by measuring the absorbance using a spectrometer at a wave-length of 338 nm and then compared to a standard curve prepared with known amounts of tetracaine. By comparing the amount of tetracaine recovered from the total oil fraction to what was added to the oil bodies, it was determined that an average of 90.8% of the tetracaine added was incorporated into the intact oil bodies. The level of tetracaine incorporated into the oil bodies as a percentage of dry weight was 2.43%. When loading larger volumes of oil bodies, the Cito-unguator lab mixer (Gako Konietzko) was found to be particularly efficient at mixing the oil with the oil bodies thus promoting efficient loading.

### Example 7

Partitioning of Phosphatidylcholine into Washed Safflower Oil Bodies.

Washed safflower oil bodies were prepared as described in example 1. Oil bodies were preserved with 0.1% Neolone 950 and 0.1% glycacil L. Phosphatidylcholine (PC, Supplier-Sigma) was weighed (1-300 mg) into a clean and dry 16×100 mm screw-cap Pyrex test tube. A fluorescent tracer (phosphatidic acid, Supplier-Molecular Probes) was added to the PC. The amount used was 0.05-0.25% the weight of the PC of a 1 mg/ml solution of tracer (resuspended following the manufacturer's instructions). 200 ul of isopropanol was added for every 10 mg of PC to dissolve the PC/tracer mixture. Buffer (50 mM NaH₂PO₄, pH 8.0) was added to the isopropanol mixture using the same buffer (50 mM NaH₂PO₄, pH 8.0) as the oilbodies were made in. The volume was equivalent to the volume of oilbodies being loaded. The PC/tracer/isopropanol/buffer mixture was sonicated on high for 15-30 seconds. The solvent was evaporated using a steady stream of nitrogen with gentle heating (42° C.) of the sample for 15 minutes. The oilbodies were added and the mixture incubated at 37° C. for several hours to several days in an airtight container. The oilbodies were washed twice with buffer to remove unincorporated PC. Both the wash fractions and the oilbody fractions were assessed for fluorescence quantities after extraction with hexane using a total oil extraction procedure. The total oil extraction was done by adding 4 mls of a 3:2 hexane:isopropanol solution (HIP) to a measured amount of the sample and shaking to mix vigorously until all of the oil was dissolved in the HIP solvent (about 10-20 seconds). 2.5 ml of 6.67% Na₂SO₄ (w/v) was added to the tube and the tube was shaken for another 10 seconds. Phase separation is facilitated by centrifugation for 2 minutes at 3220×g in a swinging bucket clinical centrifuge. The organic or upper phase is removed to a second test tube using a Pasteur pipette while avoiding the transfer of the aqueous phase. 3 ml of a 7:2 HIP solution was added to the original tube containing the aqueous phase and the tube shaken for 10 seconds. The tube was centrifuged for 2 minutes at 3220×g and the upper phase is combined with the organic phase retrieved in the first HIP extraction. The 7:2 HIP extraction step was repeated. The solvent was evaporated from the lipid extract by subjecting the tube containing the combined organic phases to a gently stream of compressed N₂ gas while heating (40-45° C.) in a dry block heater. The tube is weighed after one hour and then every 15 minutes after that. When two successive weights are the same (±0.0001 g), then it is assumed that all volatile components have evaporated and that only extracted lipids remain. A known quantity of isopropanol was added to the extracted fractions and the sample was quantitated using a fluorescent spectrophotometer. By comparing the amount of fluorescence recovered from the oilbody and wash fractions to what was added to the oil bodies as the tracer, it was determined that the amount of PC equivalent to an average of 0.12% of the dry weight of the oil body could be loaded in to the membrane of the oil body. Stearylamine is derived from stearic acid (beef tallow derived) and ammonia. It is a positively charged inducing agent which has been used to modify the charge of PC liposomes (Moncelli et al. (1994) Biophys. J. 66: 1969-1980). If stearylamine (Sigma) was included in the PC/tracer mixture at an amount approximately 30% of the amount of PC used for loading, then the amount of PC was equivalent to an average of 1.25% of the dry weight of the oil body could be loaded in to the membrane of the oil body.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

What we claim as our invention is:

1. A method of partitioning an active agent into oil bodies, said method comprising the steps of
a) dissolving the active agent in a first solvent;
b) mixing the dissolved active agent with a second solvent to obtain a mixture of the first and second solvent comprising the active agent; and
c) contacting said mixture of the first and second solvent with oil bodies to partition said active agent into said oil bodies.

2. A method according to claim 1 where said active agent does not partition into oil bodies when contacted with oil bodies in the absence of a solvent or when the active agent is dissolved in the first solvent alone.

3. The method according to claim 2 wherein the active agent is insoluble in water.

4. The method according to claim 2 wherein the active agent is insoluble in the second solvent.

5. A method according to claim 1 wherein the amount of said active agent partitioned in said oil bodies ranges from about 0.0001% to 50% (w/v).

6. A method according to claim 1 wherein the amount of said active agent partitioned into said oil bodies ranges from about 0.1% to 20% (w/v).

7. A method according to claim 1 wherein the amount of said active agent partitioned into said oil bodies ranges from about 0.1% to 10% (w/v).

8. A method according to claim 1 wherein the efficiency of partitioning of the,active into intact oil bodies ranges from about 10-99%.

9. A method according to claim 1 wherein the efficiency of partitioning of the active into intact oil bodies ranges from about 50-99%.

10. A method according to claim 1 wherein the efficiency of partitioning of the active into intact oil bodies ranges from about 90-99%.

11. A method according to claim 1 wherein said active agent is selected from the group of active agents consisting of hydrophobic molecules and amphipathic molecules.

12. A method according to claim 11 wherein said hydrophobic molecule is selected from the group consisting of clobetasol propionate, diclofenac, dithranol, retinoic acid, lidocaine, clindamycin, benzoyl peroxide and cyclosporine A.

13. A method according to claim 11 wherein said hydrophobic molecule has a log P value ranging from about 0 to 8.

14. A method according to claim 11 wherein said hydrophobic molecule has a log P value ranging from about 2 to 7.

15. A method according to claim 11 wherein said hydrophobic molecule has a log P value ranging from about 3 to 7.

16. A method according to claim 11 wherein said amphipathic molecule is selected from the group consisting of amphotericin B, phosphatidyl choline, tetracaine and actinomycin D.

17. A method according to claim 11 wherein said amphipathic molecule has a HLB value ranging from about 1 to 14.

18. A method according to claim 11 wherein said amphipathic molecule has a HLB value ranging from about 4 to 10.

19. A method according to claim 11 wherein said amphipathic molecule has a HLB value ranging from about 6 to 8.

20. A method according to claim 1 wherein said first solvent is non-compatible with oil bodies or undesirable in the final product.

21. A method according to claim 1 wherein said first solvent is an organic solvent.

22. A method according to claim 1 wherein said first solvent is selected from the group of solvents consisting of an alcohols, aliphatic hydrocarbons, aromatic hydrocarbons, chlorinated hydrocarbons, glycols, glycol ethers and their acetates, esters, ethers, ketones, oil, lipid and fatty acid.

23. A method according to claim 22 wherein the first solvent is an alcohol or a chlorinated hydrocarbon.

24. A method according to claim 22 wherein the first solvent is selected from the group consisting of isopropanol, ethanol and chloroform.

25. A method according to claim 1 wherein said second solvent is selected from the group of solvents consisting of water, aqueous buffer, oils, fatty acids, and lipids.

26. A method according to claim 25 wherein said aqueous buffer is selected from the group consisting of 50 mM monobasic sodium phosphate, pH 8.0 and 25 mM sodium bicarbonate, pH 8.3.

27. A method according to claim 25 wherein said oil is safflower oil.

28. A method according to claim 1 wherein said first solvent is substantially removed after it has been mixed with the second solvent.

29. A method according to claim 26 wherein said first solvent is substantially removed by evaporation or substantially reduced in volume by dilution.

30. A method according to claim 27 wherein the method of evaporation is exposing the sample to a stream of nitrogen.

31. A method according to claim 1 wherein said oil bodies are obtained from a cell containing oil bodies or oil body-like organelles.

32. A method according to claim 29 wherein said cell includes animal cells, plant cells, fungal cells, yeast cells, bacterial cells and algae cells.

33. A method according to claim 30 wherein said plant cell includes cells from pollens, spores, seed and vegetative plant organs.

34. A method according to claim 31 wherein said plant seeds are obtained from the group of plant species consisting of rapeseed (Brassica spp.), soybean (Glycine max), sunflower (Helianthus annuus), oil palm (Elaeis guineeis), cottonseed (Gossypium spp.), groundnut (Arachis hypogaea), coconut (Cocus nucifera), castor (Ricinus communis), olive (Olea spp.), safflower (Carthamus tinctorius), mustard (Brassica spp. and Sinapis alba), coriander (Coriandrum sativum), squash (Cucurbita maxima), linseed/flax (Linum usitatissimum), Brazil nut (Bertholletia excelsa), jojoba (Simmondsia chinensis), maize (Zea mays), crambe (Crambe abyssinica) and eruca (Eruca sativa).


SEQUENCE LISTING (1 sequence)

SEQ ID NO: 1 - protein, 5 aa, Artificial sequence
  peptide
  MOD_RES (4)..(4): MeGly
  MOD_RES (5)..(5): MeVal
TVPXX